(19) United States
(12) Patent Application Publication
(10) Pub. No.: US 2008/0315137 A1
(43) Pub. Date: Dec. 25, 2008

(54) X-RAY IMAGING APPARATUS

(75) Inventor: GA-LANE CHEN, Santa Clara, CA (US)

Correspondence Address: PCE INDUSTRY, INC.;ATT. CHENG-JU CHIANG, 458 E. LAMBERT ROAD, FULLERTON, CA, 92835, US

(73) Assignee: HON HAI PRECISION INDUSTRY CO., LTD., Tu-Cheng (TW)

(21) Appl. No.: 11/951,168

(22) Filed: Dec. 5, 2007

(30) Foreign Application Priority Data
Jun. 22, 2007 (CN) 200710200856.3

(51) Int. Cl. A61B 6/00

(52) U.S. Cl. 250/582

(57) ABSTRACT

An exemplary X-ray imaging apparatus includes an X-ray source, a phosphor layer, an optical leveling element, an image sensor, an image processor, and a display. The optical leveling element includes a plurality of lenses formed thereon. A refractive index of the plurality of the lenses progressively increases from a center of the optical leveling element to a periphery of the optical leveling element. The X-ray source is configured for emitting X-rays towards an object. The phosphor layer is configured for converting the X-rays to visible light. The phosphor layer is disposed between the X-ray source and the optical leveling element. The image sensor is configured for sensing the visible light passing through the optical leveling element, and thus capturing an image of the object. The display is electrically connected with the image processor and configured for displaying the image of the object.

### BACKGROUND

1. Technical Field

The present invention relates to X-ray imaging field and, particularly, to an X-ray imaging apparatus.

2. Description of Related Art

X-ray imaging apparatuses have been widely used in medical imaging. Generally, the X-ray imaging apparatuses use photographic films to obtain images.

However, sizes of particles used in the photographic films are limited, and it is difficult to further overcome unevenness problem in particle sizes. Accordingly, the photographic films limit further improvement of imaging quality. Furthermore, it is inconvenient and time consuming using photographic films to obtain image.

Therefore, a new X-ray imaging apparatus is desired to overcome the above mentioned problems.

### SUMMARY

An exemplary X-ray imaging apparatus includes an X-ray source, a phosphor layer, an optical leveling element, an image sensor, an image processor, and a display. The optical leveling element includes a plurality of lenses formed thereon. A refractive index of the plurality of the lenses progressively increases from a center of the optical leveling element to a periphery of the optical leveling element. The X-ray source is configured for emitting X-rays towards an object. The phosphor layer is configured for converting the X-rays to visible light. The phosphor layer is disposed between the X-ray source and the optical leveling element. The image sensor is configured for sensing the visible light passing through the optical leveling element, and thus capturing an image of the object. The display is electrically connected with the image processor and configured for displaying the image of the object.

### BRIEF DESCRIPTION OF THE DRAWINGS

Many aspects of the embodiment can be better understood with references to the following drawings. The components in the drawings are not necessarily drawn to scale, the emphasis instead being placed upon clearly illustrating the principles of the present embodiments. Moreover, in the drawings, like reference numerals designate corresponding parts throughout the several views.

FIG. 1 is a schematic, side cross-sectional view of an X-ray imaging apparatus according to a present embodiment.

FIG. 2 is a schematic, exploded view of a collimating lens, an optical leveling element, and an image sensor of FIG. 1.

FIG. 3 is a side elevation optical path view of a lens at a center of the optical leveling element of FIG. 1, together with a corresponding part of the image sensor.

FIG. 4 is a side elevation optical path view of a lens at a periphery of the optical leveling element of FIG. 1, together with a corresponding part of the image sensor.

### DETAILED DESCRIPTION OF THE EMBODIMENTS

Embodiments will now be described in detail below with reference to the drawings.

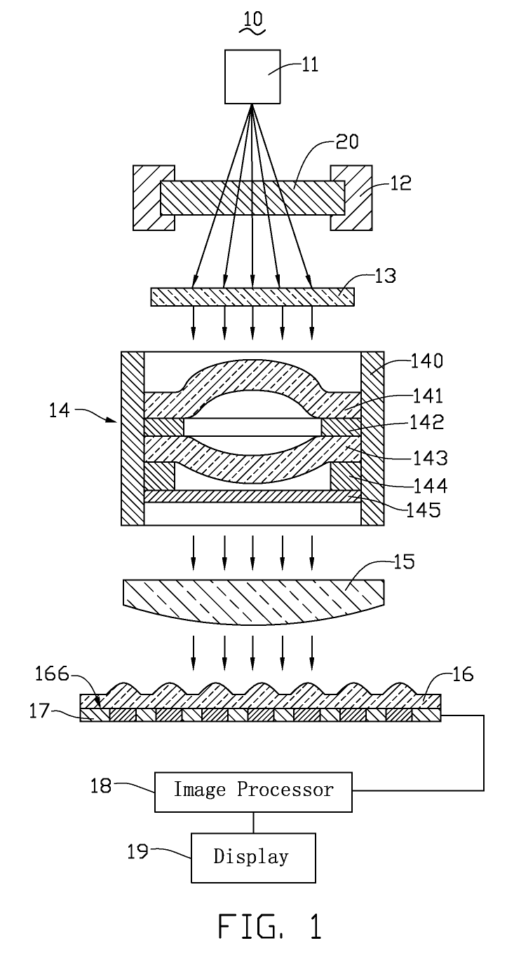

Referring to FIG. 1, an X-ray imaging apparatus 10 of an exemplary embodiment is shown. The X-ray imaging apparatus 10 includes an X-ray source 11, a holder 12, a phosphor layer 13, a lens module 14, a collimating lens 15, an optical leveling element 16, an image sensor 17, an image processor 18, and a display 19 in the order written. The image sensor 17 is electrically connected with the image processor 18, and the image processor 18 is electrically connected with the display 19.

A metal target of the X-ray source 11 can be made of copper. A working voltage of the X-ray source 11 can be in an approximate range from 10 kilovolts (KV) to 100 KV, and generally in an approximate range from 20 KV to 60 KV. A working current of the X-ray source can be in an approximate range from 0.01 milliamperes (mA) to 1 mA, and generally in an approximate range from 0.05 mA to 0.5 mA.

The holder 12 is configured for supporting an object 20. The object 20 can be a human body. The holder 12 can have several degrees of freedom as needed. For instance, in order to obtain stereo image of the object 20, the holder 12 can have six degrees of freedom (i.e., three translation degrees of freedom and three rotation degrees of freedom). That is, the holder is capable of translating along X axis, Y axis, Z axis, and rotating around the X axis, the Y axis, the Z axis in a Cartesian coordinate system. The phosphor layer 13 is configured for converting the X-ray to visible light.

The lens module 14 includes a barrel 140, a first lens 141, a second lens 143, an infrared-cut filter 145, a first spacer 142, and a second spacer 144 received in the barrel 140. The first lens 141 and the second lens 143 can be aspherical lenses. The aspherical lens's more complex surface profile can eliminate spherical aberration and reduce other optical aberrations compared to a simple lens. The infrared-cut filter 145 is configured for blocking infrared wavelengths, thus reducing thermal noise stimulated by infrared wavelengths to the image sensor 17. In the present embodiment, the lens module 14 includes two lenses. It should be noted that the lens module 14 can include more than two lenses if needed.

The image sensor 17 can be a charge coupled device (CCD) or a complementary metal oxide semiconductor (CMOS). The image sensor 17 includes a plurality of sensitive regions (e.g., 172 and 174) and a plurality of insensitive regions (not labeled). The insensitive regions are positioned between the sensitive regions. The display 19 can be a thin film transistor liquid crystal display (TFT-LCD).

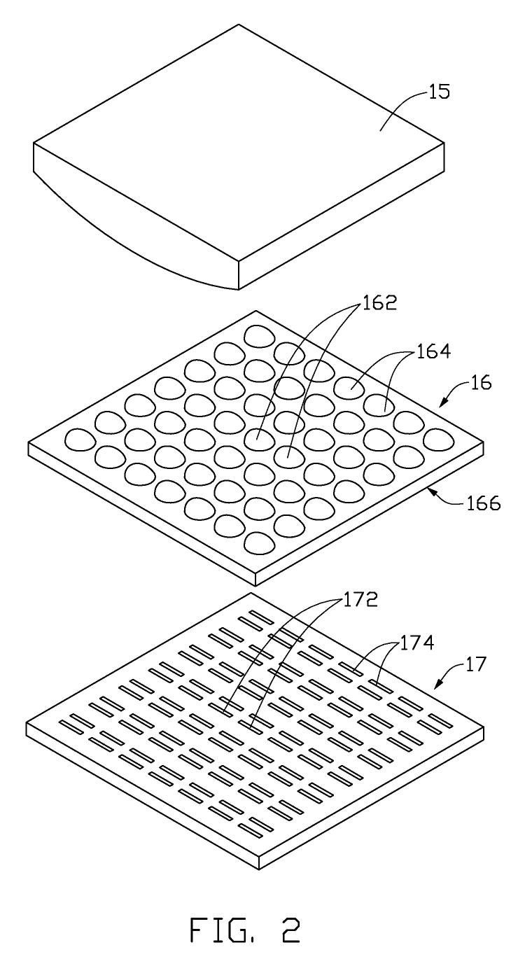

Referring to FIGS. 1 and 2, the optical leveling element 16 has a plurality of lenses (e.g., 162 and 164) spatially corresponding to the sensitive regions of the image sensor 17. A method for making an optical leveling element 16 is taught in U.S. Pat. No. 6,950,238 entitled “Optical leveling module and method for manufacturing an optical leveling layer thereof”, which is incorporated herein by reference. The plurality of the lenses are distributed in rows and columns. The lenses have different refractive indexes, which gradually vary from low at the center of the optical leveling element 16 to high at the periphery of the optical leveling element 16. Each of the plurality of the lenses can have a spherical surface or an aspherical surface.

The collimating lens 15 is configured for attaining parallelism of the visible light emitting therefrom. The visible light emit along a vertical direction from the collimating lens 15. The “vertical direction” is a direction perpendicular to a bottom surface 166 of the optical leveling element 16.

Referring to FIG. 1 again, in operation, X-ray source 11 emits X-rays, and then the X-rays pass through the object 20. The X-rays strike the phosphor layer 13, and the phosphor layer 13 converts the X-rays to visible light. The visible light goes through the lens module, and then emits from the collimating lens 15 in the vertical direction by the collimating lens 15. The visible light from the collimating lens 15 is refracted by the optical leveling element 16 and then reaches the sensitive regions of the image sensor 17. The image sensor 17 senses the visible light and then generates a first signal in response to the visible light. The image sensor 17 sends the first signal to the image processor 18. The image processor 18 treats the first signal, obtains a second signal, and then sends the second signal to the display 19. The display 19 receives the second signal, thereby displaying an image of the object 20.

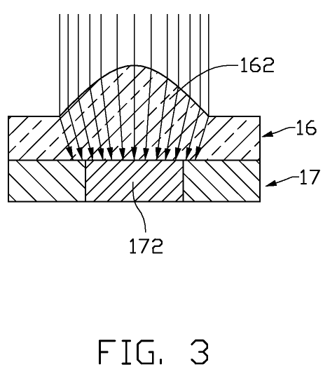

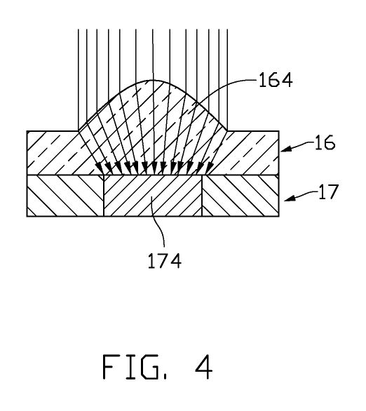

Referring to FIGS. 2-4, the optical leveling element 16 and the image sensor 17 have a certain fixed distance therebetween. An exemplary lens 162 at the center of the optical leveling element 16 has a low refractive index, and light passing therethrough focuses at a relatively distant point. An exemplary lens 164 at the periphery of the optical leveling element 16 has a high refractive index, and light passing therethrough focuses at a relatively close point. Accordingly, the sensitive region 172 receives less light flux than the sensitive region 174 because light flux from the lens 162 to the corresponding sensitive region 172 is lost (i.e., reaching the insensitive regions) much more than light flux from the lens 164 to the corresponding sensitive region 174. The lost light flux is received by the insensitive regions. Thus the optical intensity of the sensitive region 172 is weaker than that of the sensitive region 174. That is, a part of an image formed on the image sensor 17 at the center thereof is weakened, and a part of the image at the periphery of the image sensor 17 is relatively intensified. In other words, the intensity of the whole image is leveled throughout the image sensor 17.

The refractive indexes of the lenses (e.g., 162 and 164) on the optical leveling element gradually increase from the center to the periphery thereof, in order to compensate for the asymmetry of image intensity caused by vignetting and Cos θlaw (Cos θlaw defines that the peripheral image intensity is reduced in proportion to four times the cosine of the incident angle). After being adjusted by the optical leveling element 16, the intensity of the image formed on the image sensor 17 is essentially uniform, and the quality of the image is improved.

The present X-ray imaging apparatus 10 forms an image on the display 19 immediately by using the image sensor 17. Thus, the present X-ray imaging apparatus 10 is free of photographic films, and quality of the image can be improved. Furthermore, the optical leveling element 16 compensates for the asymmetry of image intensity caused by vignetting and Cos θlaw. In this way, the quality of the image is further improved.

While certain embodiments have been described and exemplified above, various other embodiments will be apparent to those skilled in the art from the foregoing disclosure. The present invention is not limited to the particular embodiments described and exemplified but is capable of considerable variation and modification without departure from the scope of the appended claims.

1. An X-ray imaging apparatus comprising:
an X-ray source configured for emitting X-rays toward an object;
an optical leveling element comprising a plurality of lenses formed thereon, wherein a refractive index of the plurality of the lenses progressively increases from a center of the optical leveling element to a periphery of the optical leveling element;
a phosphor layer configured for converting the X-rays to visible light, the phosphor layer being disposed between the X-ray source and the optical leveling element;
a lens module comprising a first lens and a second lens, the lens module being positioned between the phosphor layer and the optical leveling element, wherein visible light is capable of passing through the first lens and the second lens sequentially and then passing through the optical leveling element:
an image sensor configured for sensing the visible light passing through the optical leveling element, and capturing an image of the object; and
a display electrically connected with the image sensor, for displaying the image of the object.

2. The X-ray imaging apparatus as claimed in claim 1, wherein the image sensor comprises a plurality of sensitive regions, and each of the lenses spatially corresponds to each of the sensitive regions.

3. The X-ray imaging apparatus as claimed in claim 1, wherein the image sensor is a charged coupled device or a complementary metal oxide semiconductor.

4. The X-ray imaging apparatus as claimed in claim 1, further comprising a collimating lens disposed between the phosphor layer and the optical leveling element, the collimating lens being configured for attaining parallelism of the visible light emitting therefrom.

5. (canceled)

6. The X-ray imaging apparatus as claimed in claim 1, wherein the first lens is an aspherical lens.

7. The X-ray imaging apparatus as claimed in claim 1, wherein the lens module further comprises an infrared-cut filter.

8. The X-ray imaging apparatus as claimed in claim 1, further comprising a holder configured for supporting an object, the holder being placed between the X-ray source and the phosphor layer.

9. The X-ray imaging apparatus as claimed in claim 1, wherein the display is a liquid crystal display.

10. The X-ray imaging apparatus as claimed in claim 1, wherein each of the plurality of the lenses has a spherical or aspherical surface.

11. The X-ray imaging apparatus as claimed in claim 1, wherein the plurality of the lenses are distributed in rows and columns.

12. An X-ray imaging apparatus comprising:
an X-ray source for emitting X-rays toward an object;
a phosphor layer configured for converting the X-rays to visible light;
an image sensor configured for sensing the visible light and capturing an image of the object;
an optical leveling element disposed between the phosphor layer and the image sensor, the optical leveling element comprising a plurality of lenses formed thereon, wherein a refractive index of the plurality of the lenses progressively increases from a center of the optical leveling element to a periphery of the optical leveling; and
a lens module comprising a first lens and a second lens, the lens module being positioned between the phosphor layer and the optical leveling element, wherein the visible light is capable of passing through the first lens and the second lens sequentially and then passing through the optical leveling element.

13. The X-ray imaging apparatus as claimed in claim 12, wherein the image sensor comprises a plurality of sensitive regions, and each of the lenses spatially corresponds to each of the sensitive regions.

14. The X-ray imaging apparatus as claimed in claim 12, further comprising a collimating lens disposed between the phosphor layer and the optical leveling element, the collimating lens being configured for attaining parallelism of the visible light emitting therefrom.

15. (canceled)

16. The X-ray imaging apparatus as claimed in claim 12, wherein each of the plurality of the lenses has a spherical or aspherical surface.

17. The X-ray imaging apparatus as claimed in claim 12, wherein the plurality of the lenses are distributed in rows and columns.
